SEQUENCE LISTING (32 sequences)

SEQ ID NO: 1 - DNA, 1278 bp, Escherichia coli
atgctcgaattgctttacaccgcccttctctaccttattcagccgctgatctggatacggctctgggtgcgcggacgtaaggctccggcctatcgaaaacgctggggtgaacgttacggtttttaccgccatccgctaaaaccaggcggcattatgctgcactccgtctccgtcggtgaaactctggcggcaatcccgttggtgcgcgcgctgcgtcatcgttatcctgatttaccgattaccgtaacaaccatgacgccaaccggttcggagcgcgtacaatcggctttcgggaaggatgttcagcacgtttatctgccgtatgatctgcccgatgcactcaaccgtttcctgaataaagtcgaccctaaactggtgttgattatggaaaccgaactatggcctaacctgattgcggcgctacataaacgtaaaattccgctggtgatcgctaacgcgcgactctctgcccgctcggccgcaggttatgccaaactgggtaaattcgtccgtcgcttgctgcgtcgtattacgctgattgctgcgcaaaatgaagaagatggtgcacgttttgtggcgctgggcgcaaaaaataatcaggtgaccgttaccggtagcctgaaattcgatatttctgtaacgccgcagttggctgctaaagccgtgacgctgcgccgccagtgggcaccacaccgcccggtatggattgccaccagcactcacgaaggcgaagagagtgtggtgatcgccgcacatcaggcattgttacagcaattcccgaatttattgctcatcctggtaccccgtcatccggaacgcttcccggatgcgattaaccttgtccgccaggctggactaagctatatcacacgctcttcaggggaagtcccctccaccagcacgcaggttgtggttggcgatacgatgggcgagttgatgttactgtatggcattgccgatctcgcctttgttggcggttcactggttgaacgtggtgggcataatccgctggaagctgccgcacacgctattccggtattgatggggccgcatacttttaactttaaagacatttgcgcgcggctggagcaggcaagcgggctgattaccgttaccgatgccactacgcttgcaaaagaggtttcctctttactcaccgacgccgattaccgtagtttctatggccgtcatgccgttgaagtactgtatcaaaaccagggcgcgctacagcgtctgcttcaactgctggaaccttacctgccaccgaaaacgcattga

SEQ ID NO: 2 - DNA, 1278 bp, Artificial sequence
  synthesized
atgctcgaattgctttacaccgcccttctctaccttattcagccgctgatctggatacggctctgggtgcgcggacgtaagactccggcctatcgaaaacgctggggtgaacgttacggtttttaccgccatccgctaaaaccaggcggcattatgctgcactccgtctccgtcggtgaaactctggcggcaatcccgttggtgcgcgcgctgcgtcatcgttatcctgatttaccgattaccgtaacaaccatgacgccaaccggttcggagcgcgtacaatcggctttcgggaaggatgttcagcacgtttatctgccgtatgatctgcccgatgcactcaaccgtttcctgaataaagtcgaccctaaactggtgttgattatggaaaccgaactatggcctaacctgattgcggcgctacataaacgtaaaattccgctggtgatcgctaacgcgcgactctctgcccgctcggccgcaggttatgccaaactgggtaaattcgtccgtcgcttgctgcgtcgtattacgctgattgctgcgcaaaatgaagaagatggtgcacgttttgtggcgctgggcgcaaaaaataatcaggtgaccgttaccggtagcctgaaattcgatatttctgtaacgccgcagttggctgctaaagccgtgacgctgcgccgccagtgggcaccacaccgcccggtatggattgccaccagcactcacgaaggcgaagagagtgtggtgatcgccgcacatcaggcattgttacagcaattcccgaatttattgctcatcctggtaccccgtcatccggaacgcttcccggatgcgattaaccttgtccgccaggctggactaagctatatcacacgctcttcaggggaagtcccctccaccagcacgcaggttgtggttggcgatacgatgggcgagttgatgttactgtatggcattgccgatctcgcctttgttggcggttcactggttgaacgtggtgggcataatccgctggaagctgccgcacacgctattccggtattgatggggccgcatacttttaactttaaagacatttgcgcgcggctggagcaggcaagcgggctgattaccgttaccgatgccactacgcttgcaaaagaggtttcctctttactcaccgacgccgattaccgtagtttctatggccgtcatgccgttgaagtactgtatcaaaaccagggcgcgctacagcgtctgcttcaactgctggaaccttacctgccaccgaaaacgcattga

SEQ ID NO: 3 - protein, 425 aa, Escherichia coli
MLELLYTALLYLIQPLIWIRLWVRGRKAPAYRKRWGERYGFYRHPLKPGGIMLHSVSVGETLAAIPLVRALRHRYPDLPITVTTMTPTGSERVQSAFGKDVQHVYLPYDLPDALNRFLNKVDPKLVLIMETELWPNLIAALHKRKIPLVIANARLSARSAAGYAKLGKFVRRLLRRITLIAAQNEEDGARFVALGAKNNQVTVTGSLKFDISVTPQLAAKAVTLRRQWAPHRPVWIATSTHEGEESVVIAAHQALLQQFPNLLLILVPRHPERFPDAINLVRQAGLSYITRSSGEVPSTSTQVVVGDTMGELMLLYGIADLAFVGGSLVERGGHNPLEAAAHAIPVLMGPHTFNFKDICARLEQASGLITVTDATTLAKEVSSLLTDADYRSFYGRHAVEVLYQNQGALQRLLQLLEPYLPPKTH

SEQ ID NO: 4 - protein, 425 aa, Artificial sequence
  synthesized
MLELLYTALLYLIQPLIWIRLWVRGRKTPAYRKRWGERYGFYRHPLKPGGIMLHSVSVGETLAAIPLVRALRHRYPDLPITVTTMTPTGSERVQSAFGKDVQHVYLPYDLPDALNRFLNKVDPKLVLIMETELWPNLIAALHKRKIPLVIANARLSARSAAGYAKLGKFVRRLLRRITLIAAQNEEDGARFVALGAKNNQVTVTGSLKFDISVTPQLAAKAVTLRRQWAPHRPVWIATSTHEGEESVVIAAHQALLQQFPNLLLILVPRHPERFPDAINLVRQAGLSYITRSSGEVPSTSTQVVVGDTMGELMLLYGIADLAFVGGSLVERGGHNPLEAAAHAIPVLMGPHTFNFKDICARLEQASGLITVTDATTLAKEVSSLLTDADYRSFYGRHAVEVLYQNQGALQRLLQLLEPYLPPKTH

SEQ ID NO: 5 - DNA, 26 bp, Artificial sequence
  synthesized
cgggatccaccagtgaaccgccaaca

SEQ ID NO: 6 - DNA, 18 bp, Artificial sequence
  synthesized
tgcgcggacgtaagactc

SEQ ID NO: 7 - DNA, 18 bp, Artificial sequence
  synthesized
gagtcttacgtccgcgca

SEQ ID NO: 8 - DNA, 35 bp, Artificial sequence
  synthesized
aaggaaaaaagcggccgcttcccgcacctttattg

SEQ ID NO: 9 - DNA, 18 bp, Artificial sequence
  synthesized
cttcccgaaagccgattg

SEQ ID NO: 10 - DNA, 18 bp, Artificial sequence
  synthesized
acaaaatatactttaatc

SEQ ID NO: 11 - DNA, 1596 bp, Artificial sequence
  synthesized
accagtgaaccgccaacaaaggcgagatcggcaatgccatacagtaacatcaactcgcccatcgtatcgccaaccacaacctgcgtgctggtggaggggacttcccctgaagagcgtgtgatatagcttagtccagcctggcggacaaggttaatcgcatccgggaagcgttccggatgacggggtaccaggatgagcaataaattcgggaattgctgtaacaatgcctgatgtgcggcgatcaccacactctcttcgccttcgtgagtgctggtggcaatccataccgggcggtgtggtgcccactggcggcgcagcgtcacggctttagcagccaactgcggcgttacagaaatatcgaatttcaggctaccggtaacggtcacctgattattttttgcgcccagcgccacaaaacgtgcaccatcttcttcattttgcgcagcaatcagcgtaatacgacgcagcaagcgacggacgaatttacccagtttggcataacctgcggccgagcgggcagagagtcgcgcgttagcgatcaccagcggaattttacgtttatgtagcgccgcaatcaggttaggccatagttcggtttccataatcaacaccagtttagggtcgactttattcaggaaacggttgagtgcatcgggcagatcatacggcagataaacgtgctgaacatccttcccgaaagccgattgtacgcgctccgaaccggttggcgtcatggttgttacggtaatcggtaaatcaggataacgatgacgcagcgcgcgcaccaacgggattgccgccagagtttcaccgacggagacggagtgcagcataatgccgcctggttttagcggatggcggtaaaaaccgtaacgttcaccccagcgttttcgataggccggagacttacgtccgcgcacccagagccgtatccagatcagcggctgaataaggtagagaagggcggtgtaaagcaattcgagcatagtaaatagctgacttatggatgtgctggggattctatgtatttagctgtggctttaccattacttttcccgtttttgacttaaatagcttcagtttggtctgatctgccgctacatcttcattttttttgtatttttatgcgattcattgaaactcggccccattttcaaatctacataggccgtactgacattatcgaaatgctattttttatctatttgatttttatgattaaagtatattttgtgtataaaaatcattcgggtcggattgctgcgaaagaaatgatacactagcacgtcaaagtaagtgcgttatcagtattcaggtagctgttgagcctggggcggtagcgtgcttttttctgcttaacttaaccagacaatcacacaaaagagtcgctagtggaaaagccatttcgaaaaatcctggtcataaagatgcgatatcatggggatatgttattaactactcctgtcatcagtacgctcaagcagaattatcctgatgcaaaaatcgatatgctgctttatcaggacaccatccctattttgtctgaaaacccggaaattaatgcgctctatgggataagcaataaaggtgcgggaa

SEQ ID NO: 12 - DNA, 544 bp, Artificial sequence
  synthesized
cttcccgaaagccgattgtacgcgctccgaaccggttggcgtcatggttgttacggtaatcggtaaatcaggataacgatgacgcagcgcgcgcaccaacgggattgccgccagagtttcaccgacggagacggagtgcagcataatgccgcctggttttagcggatggcggtaaaaaccgtaacgttcaccccagcgttttcgataggccggagacttacgtccgcgcacccagagccgtatccagatcagcggctgaataaggtagagaagggcggtgtaaagcaattcgagcatagtaaatagctgacttatggatgtgctggggattctatgtatttagctgtggctttaccattacttttcccgtttttgacttaaatagcttcagtttggtctgatctgccgctacatcttcattttttttgtatttttatgcgattcattgaaactcggccccattttcaaatctacataggccgtactgacattatcgaaatgctattttttatctatttgatttttatgattaaagtatattttg

SEQ ID NO: 13 - DNA, 2109 bp, Escherichia coli
atgtatctgtttgaaagcctgaatcaactgattcaaacctacctgccggaagaccaaatcaagcgtctgcggcaggcgtatctcgttgcacgtgatgctcacgaggggcaaacacgttcaagcggtgaaccctatatcacgcacccggtagcggttgcctgcattctggccgagatgaaactcgactatgaaacgctgatggcggcgctgctgcatgacgtgattgaagatactcccgccacctaccaggatatggaacagctttttggtaaaagcgtcgccgagctggtagagggggtgtcgaaacttgataaactcaagttccgcgataagaaagaggcgcaggccgaaaactttcgcaagatgattatggcgatggtgcaggatatccgcgtcatcctcatcaaacttgccgaccgtacccacaacatgcgcacgctgggctcacttcgcccggacaaacgtcgccgcatcgcccgtgaaactctcgaaatttatagcccgctggcgcaccgtttaggtatccaccacattaaaaccgaactcgaagagctgggttttgaggcgctgtatcccaaccgttatcgcgtaatcaaagaagtggtgaaagccgcgcgcggcaaccgtaaagagatgatccagaagattctttctgaaatcgaagggcgtttgcaggaagcgggaataccgtgccgcgtcagtggtcgcgagaagcatctttattcgatttactgcaaaatggtgctcaaagagcagcgttttcactcgatcatggacatctacgctttccgcgtgatcgtcaatgattctgacacctgttatcgcgtgctgggccagatgcacagcctgtacaagccgcgtccgggccgcgtgaaagactatatcgccattccaaaagcgaacggctatcagtctttgcacacctcgatgatcggcccgcacggtgtgccggttgaggtccagatccgtaccgaagatatggaccagatggcggagatgggtgttgccgcgcactgggcttataaagagcacggcgaaaccagtactaccgcacaaatccgcgcccagcgctggatgcaaagcctgctggagctgcaacagagcgccggtagttcgtttgaatttatcgagagcgttaaatccgatctcttcccggatgagatttacgttttcacaccggaagggcgcattgtcgagctgcctgccggtgcaacgcccgtcgacttcgcttatgcagtgcataccgatatcggtcatgcctgcgtgggcgcacgcgttgaccgccagccttacccgctgtcgcagccgcttaccagcggtcaaaccgttgaaatcattaccgctccgggcgctcgcccgaatgccgcttggctgaactttgtcgttagctcgaaagcgcgcgccaaaattcgtcagttgctgaaaaacctcaagcgtgatgattctgtaagcctgggccgtcgtctgctcaaccatgctttgggtggtagccgtaagctgaatgaaatcccgcaggaaaatattcagcgcgagctggatcgcatgaagctggcaacgcttgacgatctgctggcagaaatcggacttggtaacgcaatgagcgtggtggtcgcgaaaaatctgcaacatggggacgcctccattccaccggcaacccaaagccacggacatctgcccattaaaggtgccgatggcgtgctgatcacctttgcgaaatgctgccgccctattcctggcgacccgattatcgcccacgtcagccccggtaaaggtctggtgatccaccatgaatcctgccgtaatatccgtggctaccagaaagagccagagaagtttatggctgtggaatgggataaagagacggcgcaggagttcatcaccgaaatcaaggtggagatgttcaatcatcagggtgcgctggcaaacctgacggcggcaattaacaccacgacttcgaatattcaaagtttgaatacggaagagaaagatggtcgcgtctacagcgcctttattcgtctgaccgctcgtgaccgtgtgcatctggcgaatatcatgcgcaaaatccgcgtgatgccagacgtgattaaagtcacccgaaaccgaaattaa

SEQ ID NO: 14 - DNA, 2109 bp, Artificial sequence
  synthesized
atgtatctgtttgaaagcctgaatcaactgattcaaacctacctgccggaagaccaaatcaagcgtctgcggcaggcgtatctcgttgcacgtgatgctcacgaggggcaaacacgttcaagcggtgaaccctatatcacgcacccggtagcggttgcctgcattctggccgagatgaaactcgactatgaaacgctgatggcggcgctgctgcatgacgtgattgaagatactcccgccacctaccaggatatggaacagctttttggtaaaagcgtcgccgagctggtagagggggtgtcgaaacttgataaactcaagttccgcgataagaaagaggcgcaggccgaaaactttcgcaagatgattatggcgatggtgcaggatatccgcgtcatcctcatcaaacttgccgaccgtacccacaacatgcgcacgctgggctcacttcgcccggacaaacgtcgccgcatcgcccgtgaaactctcgaaatttatagcccgctggcgcaccgtttatgtatccaccacattaaaaccgaactcgaagagctgggttttgaggcgctgtatcccaaccgttatcgcgtaatcaaagaagtggtgaaagccgcgcgcggcaaccgtaaagagatgatccagaagattctttctgaaatcgaagggcgtttgcaggaagcgggaataccgtgccgcgtcagtggtcgcgagaagcatctttattcgatttactgcaaaatggtgctcaaagagcagcgttttcactcgatcatggacatctacgctttccgcgtgatcgtcaatgattctgacacctgttatcgcgtgctgggccagatgcacagcctgtacaagccgcgtccgggccgcgtgaaagactatatcgccattccaaaagcgaacggctatcagtctttgcacacctcgatgatcggcccgcacggtgtgccggttgaggtccagatccgtaccgaagatatggaccagatggcggagatgggtgttgccgcgcactgggcttataaagagcacggcgaaaccagtactaccgcacaaatccgcgcccagcgctggatgcaaagcctgctggagctgcaacagagcgccggtagttcgtttgaatttatcgagagcgttaaatccgatctcttcccggatgagatttacgttttcacaccggaagggcgcattgtcgagctgcctgccggtgcaacgcccgtcgacttcgcttatgcagtgcataccgatatcggtcatgcctgcgtgggcgcacgcgttgaccgccagccttacccgctgtcgcagccgcttaccagcggtcaaaccgttgaaatcattaccgctccgggcgctcgcccgaatgccgcttggctgaactttgtcgttagctcgaaagcgcgcgccaaaattcgtcagttgctgaaaaacctcaagcgtgatgattctgtaagcctgggccgtcgtctgctcaaccatgctttgggtggtagccgtaagctgaatgaaatcccgcaggaaaatattcagcgcgagctggatcgcatgaagctggcaacgcttgacgatctgctggcagaaatcggacttggtaacgcaatgagcgtggtggtcgcgaaaaatctgcaacatggggacgcctccattccaccggcaacccaaagccacggacatctgcccattaaaggtgccgatggcgtgctgatcacctttgcgaaatgctgccgccctattcctggcgacccgattatcgcccacgtcagccccggtaaaggtctggtgatccaccatgaatcctgccgtaatatccgtggctaccagaaagagccagagaagtttatggctgtggaatgggataaagagacggcgcaggagttcatcaccgaaatcaaggtggagatgttcaatcatcagggtgcgctggcaaacctgacggcggcaattaacaccacgacttcgaatattcaaagtttgaatacggaagagaaagatggtcgcgtctacagcgcctttattcgtctgaccgctcgtgaccgtgtgcatctggcgaatatcatgcgcaaaatccgcgtgatgccagacgtgattaaagtcacccgaaaccgaaattaa

SEQ ID NO: 15 - protein, 702 aa, Escherichia coli
MYLFESLNQLIQTYLPEDQIKRLRQAYLVARDAHEGQTRSSGEPYITHPVAVACILAEMKLDYETLMAALLHDVIEDTPATYQDMEQLFGKSVAELVEGVSKLDKLKFRDKKEAQAENFRKMIMAMVQDIRVILIKLADRTHNMRTLGSLRPDKRRRIARETLEIYSPLAHRLGIHHIKTELEELGFEALYPNRYRVIKEVVKAARGNRKEMIQKILSEIEGRLQEAGIPCRVSGREKHLYSIYCKMVLKEQRFHSIMDIYAFRVIVNDSDTCYRVLGQMHSLYKPRPGRVKDYIAIPKANGYQSLHTSMIGPHGVPVEVQIRTEDMDQMAEMGVAAHWAYKEHGETSTTAQIRAQRWMQSLLELQQSAGSSFEFIESVKSDLFPDEIYVFTPEGRIVELPAGATPVDFAYAVHTDIGHACVGARVDRQPYPLSQPLTSGQTVEIITAPGARPNAAWLNFVVSSKARAKIRQLLKNLKRDDSVSLGRRLLNHALGGSRKLNEIPQENIQRELDRMKLATLDDLLAEIGLGNAMSVVVAKNLQHGDASIPPATQSHGHLPIKGADGVLITFAKCCRPIPGDPIIAHVSPGKGLVIHHESCRNIRGYQKEPEKFMAVEWDKETAQEFITEIKVEMFNHQGALANLTAAINTTTSNIQSLNTEEKDGRVYSAFIRLTARDRVHLANIMRKIRVMPDVIKVTRNRN

SEQ ID NO: 16 - protein, 702 aa, Escherichia coli
MYLFESLNQLIQTYLPEDQIKRLRQAYLVARDAHEGQTRSSGEPYITHPVAVACILAEMKLDYETLMAALLHDVIEDTPATYQDMEQLFGKSVAELVEGVSKLDKLKFRDKKEAQAENFRKMIMAMVQDIRVILIKLADRTHNMRTLGSLRPDKRRRIARETLEIYSPLAHRLCIHHIKTELEELGFEALYPNRYRVIKEVVKAARGNRKEMIQKILSEIEGRLQEAGIPCRVSGREKHLYSIYCKMVLKEQRFHSIMDIYAFRVIVNDSDTCYRVLGQMHSLYKPRPGRVKDYIAIPKANGYQSLHTSMIGPHGVPVEVQIRTEDMDQMAEMGVAAHWAYKEHGETSTTAQIRAQRWMQSLLELQQSAGSSFEFIESVKSDLFPDEIYVFTPEGRIVELPAGATPVDFAYAVHTDIGHACVGARVDRQPYPLSQPLTSGQTVEIITAPGARPNAAWLNFVVSSKARAKIRQLLKNLKRDDSVSLGRRLLNHALGGSRKLNEIPQENIQRELDRMKLATLDDLLAEIGLGNAMSVVVAKNLQHGDASIPPATQSHGHLPIKGADGVLITFAKCCRPIPGDPIIAHVSPGKGLVIHHESCRNIRGYQKEPEKFMAVEWDKETAQEFITEIKVEMFNHQGALANLTAAINTTTSNIQSLNTEEKDGRVYSAFIRLTARDRVHLANIMRKIRVMPDVIKVTRNRN

SEQ ID NO: 17 - DNA, 28 bp, Artificial sequence
  synthesized
cgggatccgaacagcaagagcaggaagc

SEQ ID NO: 18 - DNA, 19 bp, Artificial sequence
  synthesized
tgtggtggatacataaacg

SEQ ID NO: 19 - DNA, 20 bp, Artificial sequence
  synthesized
gcaccgtttatgtatccacc

SEQ ID NO: 20 - DNA, 38 bp, Artificial sequence
  synthesized
aaggaaaaaagcggccgcacgacaaagttcagccaagc

SEQ ID NO: 21 - DNA, 20 bp, Artificial sequence
  synthesized
ctttcgcaagatgattatgg

SEQ ID NO: 22 - DNA, 20 bp, Artificial sequence
  synthesized
cacggtattcccgcttcctg

SEQ ID NO: 23 - DNA, 567 bp, Escherichia coli
atgaatatcactgctactgttcttcttgcgtttggtatgtcgatggatgcatttgctgcatcaatcggtaaaggtgccaccctccataaaccgaaattttctgaagcattgcgaaccggccttatttttggtgccgtcgaaaccctgacgccgctgatcggctggggaatgggcatgttagccagccggtttgtccttgaatggaaccactggattgcgtttgtgctgctgatattcctcggcgggcgaatgattattgagggttttcgtggcgcagatgatgaagatgaagagccgcgccgtcgacacggtttctggctactggtaaccaccgcgattgccaccagcctggatgccatggctgtgggtgttggtcttgctttcctgcaggtcaacattatcgcgaccgcattggccattggttgtgcaaccttgattatgtcaacattagggatgatggttggtcgctttatcggctcaattattgggaaaaaagcggaaattctcggcgggctggtgctgatcggcatcggcgtccagatcctctggacgcacttccacggttaa

SEQ ID NO: 24 - DNA, 567 bp, Artificial sequence
  synthesized
atgaatatcactgctactgttcttcttgcgtttggtatgtcgatggatgcatttgctgcatcaatcggtaaagatgccaccctccataaaccgaaattttctgaagcattgcgaaccggccttatttttggtgccgtcgaaaccctgacgccgctgatcggctggggaatgggcatgttagccagccggtttgtccttgaatggaaccactggattgcgtttgtgctgctgatattcctcggcgggcgaatgattattgagggttttcgtggcgcagatgatgaagatgaagagccgcgccgtcgacacggtttctggctactggtaaccaccgcgattgccaccagcctggatgccatggctgtgggtgttggtcttgctttcctgcaggtcaacattatcgcgaccgcattggccattggttgtgcaaccttgattatgtcaacattagggatgatggttggtcgctttatcggctcaattattgggaaaaaagcggaaattctcggcgggctggtgctgatcggcatcggcgtccagatcctctggacgcacttccacggttaa

SEQ ID NO: 25 - protein, 188 aa, Escherichia coli
MNITATVLLAFGMSMDAFAASIGKGATLHKPKFSEALRTGLIFGAVETLTPLIGWGMGMLASRFVLEWNHWIAFVLLIFLGGRMIIEGFRGADDEDEEPRRRHGFWLLVTTAIATSLDAMAVGVGLAFLQVNIIATALAIGCATLIMSTLGMMVGRFIGSIIGKKAEILGGLVLIGIGVQILWTHFHG

SEQ ID NO: 26 - protein, 188 aa, Artificial sequence
  synthesized
MNITATVLLAFGMSMDAFAASIGKDATLHKPKFSEALRTGLIFGAVETLTPLIGWGMGMLASRFVLEWNHWIAFVLLIFLGGRMIIEGFRGADDEDEEPRRRHGFWLLVTTAIATSLDAMAVGVGLAFLQVNIIATALAIGCATLIMSTLGMMVGRFIGSIIGKKAEILGGLVLIGIGVQILWTHFHG

SEQ ID NO: 27 - DNA, 28 bp, Artificial sequence
  synthesized
cgggatcccttcgccaatgtctggattg

SEQ ID NO: 28 - DNA, 20 bp, Artificial sequence
  synthesized
atggagggtggcatctttac

SEQ ID NO: 29 - DNA, 20 bp, Artificial sequence
  synthesized
tgcatcaatcggtaaagatg

SEQ ID NO: 30 - DNA, 38 bp, Artificial sequence
  synthesized
aaggaaaaaagcggccgccaactccgcactctgctgta

SEQ ID NO: 31 - DNA, 19 bp, Artificial sequence
  synthesized
ccatcacggcttgttgttc

SEQ ID NO: 32 - DNA, 19 bp, Artificial sequence
  synthesized
acgaaaaccctcaataatc

(19) United States
(12) Patent Application Publication
(10) Pub. No.: US 2022/0324919 A1
(43) Pub. Date: Oct. 13, 2022

(54) ESCHERICHIA COLI-BASED RECOMBINANT STRAIN, CONSTRUCTION METHOD THEREFOR AND USE THEREOF

(71) Applicant: HEILONGJIANG EPPEN BIOTECH CO., LTD., Daqing, Heilongjiang (CN)

(72) Inventors: Aiying WEI, Daqing, Heilongjiang (CN); Gang MENG, Daqing, Heilongjiang (CN); Huiping JIA, Daqing, Heilongjiang (CN); Xiaohang GAO, Daqing, Heilongjiang (CN); Fengyong MA, Daqing, Heilongjiang (CN); Xiaoqun ZHOU, Daqing, Heilongjiang (CN); Chunguang ZHAO, Daqing, Heilongjiang (CN); Lipeng YANG, Daqing, Heilongjiang (CN); Houbo SU, Daqing, Heilongjiang (CN)

(21) Appl. No.: 17/753,368

(22) PCT Filed: Aug. 27, 2020
(86) PCT No.: PCT/CN2020/111842

§ 371 Date: Feb. 28, 2022

(30) Foreign Application Priority Data
Aug. 28, 2019 (CN) 201910804679.2
Aug. 28, 2019 (CN) 201910804688.1
Sep. 27, 2019 (CN) 201910926295.8

(51) Int. Cl. C07K 14/245; C12N 15/52; C12N 15/70; C12N 9/10; C12N 9/12; C12N 9/16; C12P 13/08

(52) U.S. Cl. CPC C07K 14/245; C12N 15/52; C12N 15/70; C12N 9/1081; C12N 9/1235; C12N 9/16; C12P 13/08; C12Y 204/99012; C12Y 207/06005; C12Y 301/07002; C12R 2001/19

(57) ABSTRACT

An Escherichia coli-based kdtA-gene-modified recombinant strain, a construction method therefor and use thereof are provided. A mutant gene obtained by subjecting a wild-type kdtA gene (ORF sequence is shown in a sequence 73556-74833 in GenBank accession No. CP032667.1), a wild-type spoT gene (ORF sequence is shown in a sequence 3815907-3818015 in GenBank accession No. AP009048.1) and a wild-type yebN gene (ORF sequence is shown in a sequence 1907402-1907968 in GenBank accession No. AP009048.1) of an E. coli K12 strain and a derivative strain thereof (such as MG1655 and W3110) to site-directed mutagenesis, and a recombinant strain obtained therefrom can be used for the production of L-threonine. Compared with an unmutated wild-type strain, the obtained strain can produce L-threonine with a higher concentration and has good strain stability, and also has lower production cost as an L-threonine production strain.

The present application claims priority to Chinese Patent Application No. 2019109262958 filed with China National Intellectual Property Administration on Sep. 27, 2019, and Chinese Patent Application No. 2019108046792 filed on Aug. 28, 2019, as well as Chinese Patent Application No. 2019108046881 filed on Aug. 28, 2019, the contents of which are incorporated herein by reference in their entireties.

### TECHNICAL FIELD

The present disclosure relates to the technical field of genetic engineering and microorganisms, and in particular to a kdtA-gene-modified recombinant strain, a construction method therefor and use thereof.

### BACKGROUND

L-threonine is one of the eight essential amino acids, and is an amino acid that humans and animals cannot synthesize on their own. L-threonine can strengthen the absorption of grains, regulate the metabolism balance in vivo and promote the growth and development of organisms, and thus is widely applied to the feed, medicine and food industries.

At present, L-threonine can be produced mainly via a chemical synthesis method, a protein hydrolysis method and a microbial fermentation method, wherein the microbial fermentation method has the advantages of low production cost, high production intensity and small environmental pollution, thereby becoming the most widely applied method for industrially producing L-threonine. Various bacteria can be used for microbial fermentation production of L-threonine, such as mutants obtained by wild-type induction of Escherichia coli (E. coli), Corynebacterium, and Serratia, as production strains. Specific examples include amino acid analogue resistant mutants or various auxotrophs, such as methionine, threonine, and isoleucine. However, in the conventional mutation breeding, the strain grows slowly and generates more byproducts due to random mutation, so that a high-yield strain is not easy to obtain. Therefore, the construction of recombinant E. coli by metabolic engineering is an effective way to produce L-threonine. At present, overexpression or attenuation of key enzyme genes in the amino acid synthesis pathway and the competitive pathway mediated by expression plasmids is a main means for genetic modification of E. coli. There is still a need to develop a method for producing L-threonine more economically with a high yield. E. coli, as a host for exogenous gene expression, has the advantages of clear genetic background, simple technical operation and culture conditions and economic large-scale fermentation, and thus is paid more attention by genetic engineering experts. The genome DNA of E. coli is a circular molecule in a nucleoid, and a plurality of circular plasmid DNAs can also be provided. A nucleoid in cells of E. coli has one DNA molecule with a length of about 4,700,000 base pairs, and have about 4400 genes distributed on the DNA molecule, with each gene having an average length of about 1000 base pairs. For the strains of E. coli commonly used in molecular biology, the most commonly used strains in DNA recombination experiments, with a few exceptions, are an E. coli K12 strain and a derivative thereof.

### SUMMARY

The present disclosure provides an Escherichia coli strain K12-based recombinant strain or a derivative strain thereof, a recombinant construction method therefor and use thereof in the fermentation production of an amino acid.

The present disclosure focuses on a wild-type kdtA gene (ORF sequence is shown in a sequence 73556-74833 in GenBank accession No. CP032667.1), a wild-type spoT gene (ORF sequence is shown in a sequence 3815907-3818015 in GenBank accession No. AP009048.1) and a wild-type yebN gene (ORF sequence is shown in a sequence 1907402-1907968 in GenBank accession No. AP009048.1) of an E. coli K12 strain and a derivative strain thereof (such as MG1655 and W3110), and finds that a mutant gene obtained by subjecting the gene to site-directed mutagenesis and a recombinant strain comprising the gene can be used for the production of L-threonine, and compared with an unmutated wild-type strain, the obtained strain can greatly improve the yield of L-threonine and has good strain stability, and also has lower production cost as an L-threonine production strain. Based on the above disclosures, the present disclosure provides the following three technical solutions:

For the first technical solution, provided is a nucleotide sequence comprising a sequence formed by a mutation occurring at the 82^{th} base of a coding sequence of a wild-type kdtA gene shown in SEQ ID NO: 1.

According to the present disclosure, the mutation refers to a change in a base/nucleotide at the site, and the mutation method may be at least one selected from mutagenesis, PCR site-directed mutagenesis, and/or homologous recombination, and the like.

According to the present disclosure, the mutation is that guanine (G) mutates to adenine (A) at the 82^{th} base in SEQ ID NO: 1; specifically, the mutated nucleotide sequence is shown in SEQ ID NO: 2.

The present disclosure also provides a recombinant protein encoded by the above-mentioned nucleotide sequence.

The recombinant protein disclosed herein comprises an amino acid sequence shown in SEQ ID NO: 4.

The present disclosure also provides a recombinant vector comprising the above-mentioned nucleotide sequence or the recombinant protein.

The recombinant vector disclosed herein is constructed by introducing the above-mentioned nucleotide sequence into a plasmid; as an embodiment, the plasmid is a pKOV plasmid. Specifically, the nucleotide sequence and the plasmid may be digested with an endonuclease to form complementary cohesive ends which are ligated to construct a recombinant vector.

The present disclosure also provides a recombinant strain, which comprises a kdtA gene coding nucleotide sequence having a point mutation occurring at the coding sequence.

The recombinant strain disclosed herein contains the above-mentioned nucleotide sequence.

As an embodiment of the present disclosure, the recombinant strain contains the nucleotide sequence shown in SEQ ID NO: 2.

As an embodiment of the present disclosure, the recombinant strain contains the amino acid sequence shown in SEQ ID NO: 4.

The recombinant strain disclosed herein is formed by introducing the above-mentioned recombinant vector into a host strain; the host strain is not particularly defined, and may be selected from a L-threonine production strain known in the art that retains the kdtA gene, for example, from Escherichia coli. As an embodiment of the present disclosure, the host strain is an E. coli K12 (W3110) strain, or an E. coli CGMCC 7.232 strain.

The recombinant strain disclosed herein takes a pKOV plasmid as a vector.

The recombinant strain according to the present disclosure may or may not further comprise other modifications.

The present disclosure also provides a construction method for a recombinant strain, which comprises the following step:

modifying a nucleotide sequence of a wild-type kdtA gene coding region shown in SEQ ID NO: 1 to enable a mutation to occur at the 82^{th} base of the sequence so as to obtain a L-threonine production recombinant strain comprising the mutated kdtA coding gene.

According to the construction method of the present disclosure, the modification comprises at least one of mutagenesis, PCR site-directed mutagenesis, and/or homologous recombination, and the like.

According to the construction method of the present disclosure, the mutation is that guanine (G) mutates to adenine (A) at the 82^{th} base in SEQ ID NO: 1; specifically, the mutated nucleotide sequence is shown in SEQ ID NO: 2.

Furthermore, the construction method comprises the following steps:
(1) modifying a nucleotide sequence of an open reading frame region of the wild-type kdtA gene shown in SEQ ID NO: 1 to enable a mutation to occur at the 82^{th} base of the sequence so as to obtain a mutated nucleotide sequence of an open reading frame region of the kdtA gene;
(2) ligating the mutated nucleotide sequence to a plasmid to construct a recombinant vector; and
(3) introducing the recombinant vector into a host strain to obtain the L-threonine production recombinant strain having a point mutation.

According to the construction method of the present disclosure, the step (1) comprises: the construction of the kdtA gene coding region having a point mutation, namely comprising synthesizing two pairs of primers for amplifying kdtA gene coding region fragments according to the kdtA gene coding sequence, and introducing the point mutation in the wild-type kdtA gene coding region (SEQ ID NO: 1) by PCR site-directed mutagenesis to obtain a nucleotide sequence (SEQ ID NO: 2) of the kdtA gene coding region having the point mutation, wherein the nucleotide sequence is marked as kdtA^{(G82A)}.

In an embodiment of the present disclosure, in the step (1), the primers are:

P1:
(SEQ ID NO: 5)
5′ CGGGATCCACCAGTGAACCGCCAACA 3′;
P2:
(SEQ ID NO: 6)
5′ TGCGCGGACGTAAGACTC 3′;
P3:
(SEQ ID NO: 7)
5′ GAGTCTTACGTCCGCGCA 3′;
and
P4:
(SEQ ID NO: 8)
5′ AAGGAAAAAAGCGGCCGCTTCCCGCACCTTTATTG 3′.

In an embodiment of the present disclosure, the step (1) comprises: using primers P1/P2 and P3/P4 for PCR amplification by taking E. coli K12 as a template to obtain two isolated DNA fragments (kdtA Up and kdtA Down) having a length of 927 bp and 695 bp and kdtA gene coding regions; and separating and purifying the two DNA fragments by agarose gel electrophoresis, and then performing overlap PCR by taking P1 and P4 as primers and taking the two DNA fragments as templates to obtain kdtA^{G82A}-Up-Down.

In an embodiment of the present disclosure, the nucleotide sequence of the kdtA^{G82A}-Up-Down has a length of 1622 bp.

In an embodiment of the present disclosure, the PCR amplification is performed as follows: denaturing at 94° C. for 30 s, annealing at 52° C. for 30 s, and extending at 72° C. for 30 s (for 30 cycles).

In an embodiment of the present disclosure, the overlap PCR amplification is performed as follows: denaturing at 94° C. for 30 s, annealing at 52° C. for 30 s, and extending at 72° C. for 60 s (for 30 cycles).

According to the construction method of the present disclosure, the step (2) comprises: the construction of the recombinant vector, namely comprising separating and purifying the kdtA^{(G82A)}-Up-Down fragment by agarose gel electrophoresis, then digesting the purified fragment and the pKOV plasmid with BamH I/Not I, and separating and purifying the digested kdtA^{(G82A)}-Up-Down fragment and the digested pKOV plasmid by agarose gel electrophoresis followed by ligation to obtain the recombinant vector pKO V-kdtA^{(G82A)}.

According to the construction method of the present disclosure, the step (3) comprises: the construction of the recombinant strain, namely comprising transforming the recombinant vector pKO V-kdtA^{(G82A)}into the host strain to obtain the recombinant strain.

In an embodiment of the present disclosure, the transformation in the step (3) is an electrotransformation process; illustratively, in the step (3), the recombinant vector is introduced into the host strain.

According to the construction method of the present disclosure, the method further comprises a step of screening the recombinant strain; illustratively, screening is performed by using a chloramphenicol culture medium.

The present disclosure also provides a recombinant strain obtained by the above-mentioned construction method.

The present disclosure also provides use of the recombinant strain in the preparation of L-threonine or the improvement of L-threonine fermentation volume.

The use of the recombinant strain in the preparation of L-threonine comprises fermenting the recombinant strain to prepare L-threonine.

For the second technical solution, provided is a nucleotide sequence comprising a nucleotide sequence formed by a mutation occurring at the 520^{th} base of the spoT gene coding sequence shown in SEQ ID NO: 13.

According to the present disclosure, the mutation refers to a change in a base/nucleotide at the site, and the mutation method may be at least one selected from mutagenesis, PCR site-directed mutagenesis, and/or homologous recombination, and the like.

According to the present disclosure, the mutation is that guanine (G) mutates to thymine (T) at the 520^{th} base in SEQ ID NO: 13; specifically, the mutated nucleotide sequence is shown in SEQ ID NO: 14.

The present disclosure provides a recombinant protein encoded by the above-mentioned nucleotide sequence.

The recombinant protein disclosed herein comprises an amino acid sequence shown in SEQ ID NO: 16; specifically, the recombinant protein comprises a substitution of glycine with cysteine at the 174^{th} site of an amino acid sequence shown in SEQ ID NO: 15.

The present disclosure provides a recombinant vector comprising the above-mentioned nucleotide sequence or the recombinant protein.

The recombinant vector disclosed herein is constructed by introducing the above-mentioned nucleotide sequence into a plasmid; as an embodiment, the plasmid is a pKOV plasmid. Specifically, the nucleotide sequence and the plasmid may be digested with an endonuclease to form complementary cohesive ends which are ligated to construct a recombinant vector.

The present disclosure further provides a recombinant strain, which comprises a spoT gene coding nucleotide sequence having a point mutation on the coding sequence, for example, a spoT gene coding nucleotide sequence shown in SEQ ID NO: 13 having a point mutation occurring at the 520^{th} base.

According to the present disclosure, the mutation is that guanine (G) mutates to thymine (T) at the 520^{th} base in SEQ ID NO: 13.

As an embodiment of the present disclosure, the recombinant strain contains the nucleotide sequence shown in SEQ ID NO: 14.

As an embodiment of the present disclosure, the recombinant strain contains the amino acid sequence shown in SEQ ID NO: 16.

The recombinant strain disclosed herein is formed by introducing the above-mentioned recombinant vector into a host strain; the host strain is not particularly defined, and may be selected from a L-threonine production strain known in the art that retains the spoT gene, for example, from Escherichia coli. As an embodiment of the present disclosure, the host strain is an E. coli K12 (W3110) strain, or an E. coli CGMCC 7.232 strain.

The recombinant strain disclosed herein takes a pKOV plasmid as a vector.

The recombinant strain according to the present disclosure may or may not further comprise other modifications.

The present disclosure provides a construction method for a recombinant strain, which comprises the following step:

modifying a nucleotide sequence of a spoT gene coding region shown in SEQ ID NO: 13 to enable a mutation to occur at the 520^{th} base of the sequence so as to obtain a recombinant strain comprising the mutated spoT coding gene.

According to the construction method of the present disclosure, the modification comprises at least one of mutagenesis, PCR site-directed mutagenesis, and/or homologous recombination, and the like.

According to the construction method of the present disclosure, the mutation is that guanine (G) mutates to thymine (T) at the 520^{th} base in SEQ ID NO: 13; specifically, the mutated nucleotide sequence is shown in SEQ ID NO: 14.

Furthermore, the construction method comprises the following steps:
(1) modifying a nucleotide sequence of an open reading frame region of the wild-type spoT gene shown in SEQ ID NO: 13 to enable a mutation to occur at the 520^{th} base of the sequence so as to obtain a mutated nucleotide sequence;
(2) ligating the mutated nucleotide sequence to a plasmid to construct a recombinant vector; and
(3) introducing the recombinant vector into a host strain to obtain the recombinant strain having a point mutation.

According to the construction method of the present disclosure, the step (1) comprises: the construction of the spoT gene coding region having a point mutation, namely comprising synthesizing two pairs of primers for amplifying spoT gene coding region fragments according to the spoT gene coding sequence, and introducing the point mutation in the wild-type spoT gene coding region (SEQ ID NO: 13) by PCR site-directed mutagenesis to obtain a nucleotide sequence (SEQ ID NO: 14) of the spoT gene coding region having the point mutation, wherein the nucleotide sequence is marked as spoT^{(G520T)}.

In an embodiment of the present disclosure, in the step (1), the primers are:

P1:
(SEQ ID NO: 17)
5′ CGGGATCCGAACAGCAAGAGCAGGAAGC 3′;
P2:
(SEQ ID NO: 18)
5′ TGTGGTGGATACATAAACG 3′;
P3:
(SEQ ID NO: 19)
5′ GCACCGTTTATGTATCCACC 3′;
and
P4:
(SEQ ID NO: 20)
5′ AAGGAAAAAAGCGGCCGCACGACAAAGTTCAGCCAAGC 3′.

In an embodiment of the present disclosure, the step (1) comprises: using primers P1/P2 and P3/P4 for PCR amplification by taking E. coli K12 as a template to obtain two isolated DNA fragments (spoT^{(G520T)}-Up and spoT^{(G520T)}-Down) having a length of 620 bp and 880 bp and spoT gene coding regions having a point mutation; and separating and purifying the two DNA fragments by agarose gel electrophoresis, and then performing overlap PCR by taking P1 and P4 as primers and taking the two DNA fragments as templates to obtain a spoT^{G520T}-Up-Down fragment.

In an embodiment of the present disclosure, the nucleotide sequence of the spoT^{(G520T)}-Up-Down fragment has a length of 1500 bp.

In an embodiment of the present disclosure, the PCR amplification is performed as follows: denaturing at 94° C. for 30 s, annealing at 52° C. for 30 s, and extending at 72° C. for 30 s (for 30 cycles).

In an embodiment of the present disclosure, the overlap PCR amplification is performed as follows: denaturing at 94° C. for 30 s, annealing at 52° C. for 30 s, and extending at 72° C. for 60 s (for 30 cycles).

According to the construction method of the present disclosure, the step (2) comprises: the construction of the recombinant vector, namely comprising separating and purifying the sp^{(G520T)}-Up-Down fragment by agarose gel electrophoresis, then digesting the purified fragment and the pKOV plasmid with BamH I/Not I, and separating and purifying the digested spoT^{(G520T)}-Up-Down fragment and the digested pKOV plasmid by agarose gel electrophoresis followed by ligation to obtain the recombinant vector pKO V-spoT^{(G520T)}.

According to the construction method of the present disclosure, the step (3) comprises: the construction of the recombinant strain, namely comprising introducing the recombinant vector pKO V-spoT^{(G520T)} into the host strain to obtain the recombinant strain.

In an embodiment of the present disclosure, the introduction in the step (3) is an electrotransformation process.

According to the construction method of the present disclosure, the method further comprises a step of screening the recombinant strain; illustratively, screening is performed by using a chloramphenicol culture medium.

The present disclosure also provides a recombinant strain obtained by the above-mentioned construction method.

The present disclosure provides use of the above-mentioned recombinant strain in the preparation of L-threonine.

The use of the nucleotide sequence, the recombinant protein, the recombinant vector and the recombinant strain in the preparation of L-threonine comprises fermenting the recombinant strain to prepare L-threonine.

For the third technical solution, provided is a nucleotide sequence comprising a nucleotide sequence formed by a mutation occurring at the 74^{th} base of a wild-type yebN gene coding sequence shown in SEQ ID NO: 23.

According to the present disclosure, the mutation is that guanine (G) mutates to adenine (A) at the 74^{th} base in SEQ ID NO: 23; specifically, the nucleotide sequence is shown in SEQ ID NO: 24. The mutation refers to a change in a base/nucleotide at the site, and the mutation method may be at least one selected from mutagenesis, PCR site-directed mutagenesis, and/or homologous recombination, and the like.

The present disclosure provides a recombinant protein encoded by the above-mentioned nucleotide sequence.

The recombinant protein disclosed herein comprises an amino acid sequence shown in SEQ ID NO: 26; specifically, the recombinant protein comprises a substitution of glycine with aspartic acid at the 25^{th} site of an amino acid sequence shown in SEQ ID NO: 25.

The present disclosure provides a recombinant vector comprising the above-mentioned nucleotide sequence or the recombinant protein.

The recombinant vector disclosed herein is constructed by introducing the above-mentioned nucleotide sequence into a plasmid; as an embodiment, the plasmid is a pKOV plasmid. Specifically, the nucleotide sequence and the plasmid may be digested with an endonuclease to form complementary cohesive ends which are ligated to construct a recombinant vector.

The present disclosure also provides a recombinant strain, which comprises a yebN gene coding nucleotide sequence having a point mutation on the coding sequence, for example, a yebN gene coding nucleotide sequence shown in SEQ ID NO: 23 having a point mutation occurring at the 74^{th} base.

According to the recombinant strain, the yebN gene coding nucleotide sequence comprises a mutation that guanine (G) mutates to adenine (A) at the 74^{th} base in SEQ ID NO: 23.

As an embodiment of the present disclosure, the recombinant strain contains the nucleotide sequence shown in SEQ ID NO: 24.

As an embodiment of the present disclosure, the recombinant strain contains the amino acid sequence shown in SEQ ID NO: 26.

The recombinant strain disclosed herein is formed by introducing the above-mentioned recombinant vector into a host strain; the host strain is not particularly defined, and may be selected from a L-threonine production strain known in the art that retains the yebN gene, for example, from Escherichia coli. As an embodiment of the present disclosure, the host strain is E. coli K12, a derivative strain thereof E. coli K12 (W3110), or an E. coli CGMCC 7.232 strain.

The recombinant strain disclosed herein takes a pKOV plasmid as a vector.

The recombinant strain according to the present disclosure may or may not further comprise other modifications.

The present disclosure provides a construction method for a recombinant strain, which comprises the following step:

modifying a nucleotide sequence of a wild-type yebN gene coding region shown in SEQ ID NO: 23 to enable a mutation to occur at the 74^{th} base of the sequence so as to obtain a recombinant strain comprising the mutated yebN coding gene.

According to the construction method of the present disclosure, the modification comprises at least one of mutagenesis, PCR site-directed mutagenesis, and/or homologous recombination, and the like.

According to the construction method of the present disclosure, the mutation is that guanine (G) mutates to adenine (A) at the 74^{th} base in SEQ ID NO: 23; specifically, the mutated nucleotide sequence is shown in SEQ ID NO: 24.

Furthermore, the construction method comprises the following steps:
(1) modifying a nucleotide sequence of an open reading frame region of the wild-type yebN gene shown in SEQ ID NO: 23 to enable a mutation to occur at the 74^{th} base of the sequence so as to obtain a mutated nucleotide sequence of the open reading frame region of the yebN gene;
(2) ligating the mutated nucleotide sequence to a plasmid to construct a recombinant vector; and
(3) introducing the recombinant vector into a host strain to obtain the recombinant strain having a point mutation.

According to the construction method of the present disclosure, the step (1) comprises: the construction of the yebN gene coding region having a point mutation, namely comprising synthesizing two pairs of primers for amplifying yebN gene coding region fragments according to the yebN gene coding sequence, and introducing the point mutation in the wild-type yebN gene coding region (SEQ ID NO: 23) by PCR site-directed mutagenesis to obtain a nucleotide sequence (SEQ ID NO: 24) of the yebN gene coding region having the point mutation, wherein the nucleotide sequence is marked as yebN^{(G74A)}.

In an embodiment of the present disclosure, in the step (1), the primers are:

P1:
(SEQ ID NO: 27)
5′ CGGGATCCCTTCGCCAATGTCTGGATTG 3′;
P2:
(SEQ ID NO: 28)
5′ ATGGAGGGTGGCATCTTTAC 3′;
P3:
(SEQ ID NO: 29)
5′ TGCATCAATCGGTAAAGATG 3′;
and
P4:
(SEQ ID NO: 30)
5′ AAGGAAAAAAGCGGCCGCCAACTCCGCACTCTGCTGTA 3′.

In an embodiment of the present disclosure, the step (1) comprises: using primers P1/P2 and P3/P4 for PCR amplification by taking E. coli K12 as a template to obtain two isolated DNA fragments (yebN^{(G74A)}-Up and yebN^{(G74A)}-Down) having a length of 690 bp and 700 bp and yebN gene coding regions having a point mutation; and separating and purifying the two DNA fragments by agarose gel electrophoresis, and then performing overlap PCR by taking P1 and P4 as primers and taking the two DNA fragments as templates to obtain a yebN^{674A}-Up-Down fragment.

In an embodiment of the present disclosure, the nucleotide sequence of the yebN^{(G74A)}-Up-Down fragment has a length of 1340 bp.

In an embodiment of the present disclosure, the PCR amplification is performed as follows: denaturing at 94° C. for 30 s, annealing at 52° C. for 30 s, and extending at 72° C. for 30 s (for 30 cycles).

In an embodiment of the present disclosure, the overlap PCR amplification is performed as follows: denaturing at 94° C. for 30 s, annealing at 52° C. for 30 s, and extending at 72° C. for 60 s (for 30 cycles).

According to the construction method of the present disclosure, the step (2) comprises: the construction of the recombinant vector, namely comprising separating and purifying the yebN^{(G74A)}-Up-Down fragment by agarose gel electrophoresis, then digesting the purified fragment and the pKOV plasmid with BamH I/Not I, and separating and purifying the digested yebN^{(G74A)}-Up-Down fragment and the digested pKOV plasmid by agarose gel electrophoresis followed by ligation to obtain the recombinant vector pKO V-yebN^{(G74A)}.

According to the construction method of the present disclosure, the step (3) comprises: the construction of the recombinant strain, namely comprising introducing the recombinant vector pKO V-yebN^{(G74A)} into the host strain to obtain the recombinant strain.

In an embodiment of the present disclosure, the introduction in the step (3) is an electrotransformation process.

According to the construction method of the present disclosure, the method further comprises a step of screening the recombinant strain; illustratively, screening is performed by using a chloramphenicol culture medium.

The present disclosure also provides a recombinant strain obtained by the above-mentioned construction method.

The present disclosure provides use of the above-mentioned nucleotide sequence, the recombinant protein, the recombinant vector and the recombinant strain in the preparation of L-threonine.

The use of the recombinant strain in the preparation of L-threonine comprises fermenting the recombinant strain to prepare L-threonine.

### DETAILED DESCRIPTION

The above-mentioned and other features and advantages of the present disclosure are explained and illustrated in more detail in the following description of examples of the present disclosure. It should be understood that the following examples are intended to illustrate the technical solutions of the present disclosure, and are not intended to limit the protection scope of the present disclosure defined in the claims and equivalents thereof in any way.

Unless otherwise indicated, the materials and reagents herein are either commercially available or can be prepared by one skilled in the art in light of the prior art.

### EXAMPLE 1

(1) Construction of Plasmid pKO V-kdtA^{(G82A)}with kdtA Gene Coding Region Having Site-Directed Mutation (G82A) (equivalent to that alanine is substituted with threonine at the 28^{th} site (A28T) in a wild-type protein-coding amino acid sequence SEQ ID NO: 3, the substituted amino acid sequence being SEQ ID NO: 4)

The 3-deoxy-D-mannose-sulfamyltransferase was encoded by a kdtA gene, and in an E. coli K12 strain and a derivative strain thereof (such as MG1655), an ORF sequence of the wild-type kdtA gene is shown in a sequence 73556-74833 in Genbank accession No. CP032667.1. Two pairs of primers for amplifying kdtA were designed and synthesized according to the sequence, and a vector was constructed for a base G mutating to a base A at the 82^{th} site in a kdtA gene coding region sequence of an original strain. The primers (synthesized by Shanghai Invitrogen Corporation) were designed as follows:

P1:
(SEQ ID NO: 5)
5′ CGGGATCCACCAGTGAACCGCCAACA 3′;
P2:
(SEQ ID NO: 6)
5′ TGCGCGGACGTAAGACTC 3′;
P3:
(SEQ ID NO: 7)
5′ GAGTCTTACGTCCGCGCA 3′;
and
P4:
(SEQ ID NO: 8)
5′ AAGGAAAAAAGCGGCCGCTTCCCGCACCTTTATTG 3′.

The construction method was as follows: using primers P1/P2 and P3/P4 for PCR amplification by taking a genome of a wild-type strain E. coli K12 as a template to obtain two DNA fragments having a length of 927 bp and 695 bp and point mutation (kdtA^{(G82A)})-Up and kdtA^{(G82A)})-Down fragments). The PCR amplification was performed as follows: denaturing at 94° C. for 30 s, annealing at 52° C. for 30 s, and extending at 72° C. for 30 s (for 30 cycles). The two DNA fragments were separated and purified by agarose gel electrophoresis, and then the two purified DNA fragments were taken as templates, and P1 and P4 were taken as primers to perform overlap PCR to obtain a fragment (kdtA^{(G82A)}-Up-Down) having a length of about 1622 bp. The overlap PCR amplification was performed as follows: denaturing at 94° C. for 30 s, annealing at 52° C. for 30 s, and extending at 72° C. for 60 s (for 30 cycles). The kdtA^{(G82A)}-Up-Down fragment was separated and purified by agarose gel electrophoresis, then the purified fragment and a pKOV plasmid (purchased from Addgene) were digested with BamH I/Not I, and the digested kdtA^{(G82A)}-Up-Down fragment and the digested pKOV plasmid were separated and purified by agarose gel electrophoresis followed by ligation to obtain a vector pKO V-kdtA^{(G82A)}. The vector pKO V-kdtA^{(G82A)} was sent to a sequencing company for sequencing and identification, the result is shown in SEQ ID NO: 11, and the vector pKO V-kdtA^{(G82A)} with the correct point mutation (kdtA^{(G82A)}) was stored for later use.

(2) Construction of Engineered Strain with kdtA^{(G82A)} having Point Mutation

A wild-type kdtA gene was reserved on chromosomes of a wild-type Escherichia coli strain E. coli K12 (W3110) and a high-yield L-threonine production strain E. coli CGMCC 7.232 (preserved in China General Microbiological Culture Collection Center). The constructed plasmid pKOV-kdtA^{(G82A)} was transferred into E. coli K12 (W3110) and E. coli CGMCC 7.232, respectively, and through allele replacement, the base G mutated to the base A at the 82^{th} site of the kdtA gene sequences in the chromosomes of the two strains. The specific process was as follows: transforming the plasmid pKO V-kdtA(G82A) into host bacterium competent cells through an electrotransformation process, and adding the cells into 0.5 mL of a SOC liquid culture medium; resuscitating the mixture in a shaker at 30° C. and 100 rpm for 2 h; coating an LB solid culture medium having a chloramphenicol content of 34 mg/mL with 100 μL of the culture solution, and culturing at 30° C. for 18 h; selecting grown monoclonal colonies, inoculating the colonies in 10 mL of an LB liquid culture medium, and culturing at 37° C. and at 200 rpm for 8 h; coating an LB solid culture medium having a chloramphenicol content of 34 mg/mL with 100 μL of the culture solution, and culturing at 42° C. for 12 h; selecting 1-5 single colonies, inoculating the colonies in 1 mL of an LB liquid medium, and culturing at 37° C. and 200 rpm for 4 h; coating an LB solid culture medium containing 10% of sucrose with 100 uL of the culture solution, and culturing at 30° C. for 24 h; selecting monoclonal colonies, and streaking the colonies on an LB solid culture medium having a chloramphenicol content of 34 mg/mL and an LB solid culture medium in a one-to-one correspondence manner; and selecting strains which grew on the LB solid culture medium and could not grow on the LB solid culture medium having the chloramphenicol content of 34 mg/mL for PCR amplification identification. The primers (synthesized by Shanghai Invitrogen Corporation) for use in PCR amplification were as follows:

P5:
(SEQ ID NO: 9)
5′ CTTCCCGAAAGCCGATTG 3′;
and
P6:
(SEQ ID NO: 10)
5′ ACAAAATATACTTTAATC 3′.

SSCP (Single-Strand Conformation Polymorphism) electrophoresis was performed on the PCR-amplified product; the amplified fragment of the plasmid pKOV-kdtA^{(G82A)} was taken as a positive control, the amplified fragment of the wild-type Escherichia coli was taken as a negative control, and water was taken as a blank control. In SSCP electrophoresis, single-stranded oligonucleotide chains having the same length but different sequence arrangements formed different spatial structures in an ice bath and also had different mobilities during electrophoresis. Therefore, the fragment electrophoresis position was not consistent with that of negative control, and a strain having a fragment electrophoresis position consistent with that of positive control is the successfully allele-replaced strain. PCR amplification was performed on the target fragment by taking the successfully allele-replaced strain as a template and using primers P5 and P6, and then the target fragment was ligated to a pMD19-T vector for sequencing. Through sequence comparison of a sequencing result, a recon formed by the base G mutating to the base A at the 82^{th} site in the kdtA gene coding region sequence is the successfully modified strain, and the sequencing result is shown in SEQ ID NO: 12. The recon derived from E. coli K12 (W3110) was named as YPThr07, and the recon derived from E. coli CGMCC 7.232 was named as YPThr 08.

### (3) Threonine Fermentation Experiment

The E. coli K12 (W3110) strain, the E. coli CGMCC 7.232 strain, and the mutant strains YPThr07 and YPThr08 were inoculated in 25 mL of a liquid culture medium described in Table 1, respectively, and cultured at 37° C. and 200 rpm for 12 h. Then, 1 mL of the resulting culture of each strain was inoculated in 25 mL of a liquid culture medium described in Table 1, and subjected to fermentation culture at 37° C. and 200 rpm for 36 h. The content of L-threonine was determined by HPLC, three replicates of each strain were taken, the average was calculated, and the results are shown in Table 2.

TABLE 1
Culture medium formula

| Component | Formula g/L |
|---|---|
| Glucose | 40 |
| Ammonium sulfate | 12 |
| Potassium dihydrogen phosphate | 0.8 |
| Magnesium sulfate heptahydrate | 0.8 |
| Ferrous sulfate heptahydrate | 0.01 |
| Manganese sulfate monohydrate | 0.01 |
| Yeast extract | 1.5 |
| Calcium carbonate | 0.5 |
| L-methionine | 0.5 |
| pH value adjusted with potassium | pH 7.0 |
| hydroxide | |

TABLE 2
Threonine fermentation results

| | Fermentation | Mean value | Multiple of |
|---|---|---|---|
| Strains | volume (g/L) | (g/L) | improvement |
| E. coli K12 (W3110) | 0.03 | 0.03 | — |
| | 0.03 | | |
| | 0.02 | | |
| YPThr07 | 2.3 | 2.4 | 83.3 |
| | 2.5 | | |
| | 2.5 | | |
| E. coli CGMCC 7.232 | 15.8 | 16.1 | — |
| | 16.2 | | |
| | 16.2 | | |
| YPThr08 | 18.1 | 18.1 | 12.4% |
| | 17.9 | | |
| | 18.4 | | |

As can be seen from the results of Table 2, the substitution of alanine at the 28^{th} site of the amino acid sequence of the kdtA gene with threonine contributes to the improvement of the yield of L-threonine for the original strain producing L-threonine with either high or low yield.

### EXAMPLE 2

(1) Construction of Plasmid pKOV-spoT^{(G520T)} with spoT Gene Coding Region Having Site-Directed Mutation (G520T) (equivalent to that glycine is substituted with cysteine at the 174^{th} site (G174C) in a protein-coding amino acid sequence SEQ ID NO: 15, the substituted amino acid sequence being SEQ ID NO: 16)

SPOT enzyme was encoded by a spoT gene, and in an E. coli K12 strain and a derivative strain thereof (such as W3110), an ORF sequence of the wild-type spoT gene is shown in a sequence 3815907-3818015 in GenBank accession No. AP009048.1. Two pairs of primers for amplifying spoT were designed and synthesized according to the sequence, and a vector was constructed for a base G mutating to a base T at the 520^{th} site in a spoT gene coding region sequence of an original strain. The primers (synthesized by Shanghai Invitrogen Corporation) were designed as follows:

P1:
(SEQ ID NO: 17)
5′ CGGGATCCGAACAGCAAGAGCAGGAAGC 3′
(the underlined part is a restriction
endonuclease cutting site BamH I);
P2:
(SEQ ID NO: 18)
5′ TGTGGTGGATACATAAACG 3′;
P3:
(SEQ ID NO: 19)
5′ GCACCGTTTATGTATCCACC 3′;
and
P4:
(SEQ ID NO: 20)
5′ AAGGAAAAAAGCGGCCGCACGACAAAGTTCAGCCAAGC 3′
(the underlined part is a restriction endonuclease
cutting site Not I).

The construction method was as follows: using primers P1/P2 and P3/P4 for PCR amplification by taking a genome of a wild-type strain E. coli K12 as a template to obtain two DNA fragments having a length of 620 bp and 880 bp and point mutation (spoT^{(G520T)}-Up and spoT^{(G520T)}-Down fragments). PCR system: 10× Ex Taq buffer 5 μL, dNTP mixture (2.5 mM each) 4 μL, Mg²⁺ (25 mM) 4 μL, primers (10 pM) 2 μL each, Ex Taq (5 U/μL) 0.25 μL, total volume 50 μL, wherein the PCR was performed as follows: denaturing at 94° C. for 30 s, annealing at 52° C. for 30 s, and extending at 72° C. for 30 s (for 30 cycles). The two DNA fragments were separated and purified by agarose gel electrophoresis, and then the two purified DNA fragments were taken as templates, and P1 and P4 were taken as primers to perform overlap PCR to obtain a fragment (spoT^{(G520T)}-Up-Down) having a length of about 1500 bp. PCR system: 10× Ex Taq buffer 5 μL, dNTP mixture (2.5 mM each) 4 μL, Mg²⁺ (25 mM) 4 μL, primers (10 pM) 2 μL each, Ex Taq (5 U/μL) 0.25 μL, total volume 50 μL, wherein the overlap PCR was performed as follows: denaturing at 94° C. for 30 s, annealing at 52° C. for 30 s, and extending at 72° C. for 60 s (for 30 cycles). The spoT^{(G520T)}-Up-Down fragment was separated and purified by agarose gel electrophoresis, then the purified fragment and a pKOV plasmid (purchased from Addgene) were digested with BamH I/Not I, and the digested spoT^{(G520T)}-Up-Down fragment and the digested pKOV plasmid were separated and purified by agarose gel electrophoresis followed by ligation with a DNA ligase to obtain a vector pKO V-spoT^{(G520T)}. The vector pKOV-spoT^{(G520T)} was sent to a sequencing company for sequencing and identification, and the vector pKOV-spoT^{(G520T)} with the correct point mutation (spoT^{(G520T)}) was stored for later use.

(2) Construction of Engineered Strain with spoT^{(G520T)} Having Point Mutation

A wild-type spoT gene was reserved on chromosomes of a wild-type Escherichia coli strain E. coli K12 (W3110) and a high-yield L-threonine production strain E. coli CGMCC 7.232 (preserved in China General Microbiological Culture Collection Center). The constructed plasmid pKOV-spoT^{(G520T)} was transferred into E. coli K12 (W3110) and E. coli CGMCC 7.232, respectively, and through allele replacement, the base G mutated to the base T at the 520^{th} site of the spoT gene sequences in the chromosomes of the two strains. The specific process was as follows: transforming the plasmid pKO V-spoT^{(G520T)} into host bacterium competent cells through an electrotransformation process, and adding the cells into 0.5 mL of a SOC liquid culture medium; resuscitating the mixture in a shaker at 30° C. and 100 rpm for 2 h; coating an LB solid culture medium having a chloramphenicol content of 34 μg/mL with 100 μL of the culture solution, and culturing at 30° C. for 18 h; selecting grown monoclonal colonies, inoculating the colonies in 10 mL of an LB liquid culture medium, and culturing at 37° C. and at 200 rpm for 8 h; coating an LB solid culture medium having a chloramphenicol content of 34 μg/mL with 100 μL of the culture solution, and culturing at 42° C. for 12 h; selecting 1-5 single colonies, inoculating the colonies in 1 mL of an LB liquid medium, and culturing at 37° C. and 200 rpm for 4 h; coating an LB solid culture medium containing 10% of sucrose with 100 uL of the culture solution, and culturing at 30° C. for 24 h; selecting monoclonal colonies, and streaking the colonies on an LB solid culture medium having a chloramphenicol content of 34 μg/mL and an LB solid culture medium in a one-to-one correspondence manner; and selecting strains which grew on the LB solid culture medium and could not grow on the LB solid culture medium having the chloramphenicol content of 34 μg/mL for PCR amplification identification. The primers (synthesized by Shanghai Invitrogen Corporation) for use in PCR amplification were as follows:

P5:
(SEQ ID NO: 21)
5′ ctttcgcaagatgattatgg 3′;
and
P6:
(SEQ ID NO: 22)
5′ cacggtattcccgcttcctg 3′.

PCR system: 10× Ex Taq buffer 5 μL, dNTP mixture (2.5 mM each) 4 μL, Mg²⁺ (25 mM) 4 μL, primers (10 pM) 2 μL each, Ex Taq (5 U/μL) 0.25 μL, total volume 50 μL, wherein the PCR amplification was performed as follows: pre-denaturation at 94° C. for 5 min, (denaturing at 94° C. for 30 s, annealing at 52° C. for 30 s, and extending at 72° C. for 90 s, for 30 cycles), and over-extending at 72° C. for 10 min. SSCP (Single-Strand Conformation Polymorphism) electrophoresis was performed on the PCR-amplified product; the amplified fragment of the plasmid pKOV-spoT^{(G520T)} was taken as a positive control, the amplified fragment of the wild-type Escherichia coli was taken as a negative control, and water was taken as a blank control. In SSCP electrophoresis, single-stranded oligonucleotide chains having the same length but different sequence arrangements formed different spatial structures in an ice bath and also had different mobilities during electrophoresis. Therefore, the fragment electrophoresis position was not consistent with that of negative control, and a strain having a fragment electrophoresis position consistent with that of positive control is the successfully allele-replaced strain. PCR amplification was performed on the target fragment by taking the successfully allele-replaced strain as a template and using primers P5 and P6, and then the target fragment was ligated to a pMD19-T vector for sequencing. Through sequence comparison of a sequencing result, a recon formed by the base G mutating to the base T at the 520^{th} site in the spoT gene coding region sequence is the successfully modified strain. The recon derived from E. coli K12 (W3110) was named as YPThr03, and the recon derived from E. coli CGMCC 7.232 was named as YPThr04.

### (3) Threonine Fermentation Experiment

The E. coli K12 (W3110) strain, the E. coli CGMCC 7.232 strain, and the mutant strains YPThr03 and YPThr04 were inoculated in 25 mL of a liquid culture medium described in Table 1, respectively, and cultured at 37° C. and 200 rpm for 12 h. Then, 1 mL of the resulting culture of each strain was inoculated in 25 mL of a liquid culture medium described in Table 1, and subjected to fermentation culture at 37° C. and 200 rpm for 36 h. The content of L-threonine was determined by HPLC, three replicates of each strain were taken, the average was calculated, and the results are shown in Table 2.

TABLE 1
Culture medium formula

| Component | Formula g/L |
|---|---|
| Glucose | 40 |
| Ammonium sulfate | 12 |
| Potassium dihydrogen phosphate | 0.8 |
| Magnesium sulfate heptahydrate | 0.8 |
| Ferrous sulfate heptahydrate | 0.01 |
| Manganese sulfate monohydrate | 0.01 |
| Yeast extract | 1.5 |
| Calcium carbonate | 0.5 |
| L-methionine | 0.5 |
| pH value adjusted with | pH 7.0 |
| potassium hydroxide | |

TABLE 2
Threonine fermentation results

| | Fermentation | Mean value | Multiple of |
|---|---|---|---|
| Strains | volume (g/L) | (g/L) | improvement |
| E. coli K12 W3110 | 0.01 | 0.02 | — |
| | 0.02 | | |
| | 0.02 | | |
| YPThr03 | 2.2 | 2.3 | 115 |
| | 2.4 | | |
| | 2.3 | | |
| E. coli CGMCC | 16.0 | 16.2 | — |
| 7.232 | 16.3 | | |
| | 16.2 | | |
| YPThr04 | 17.9 | 18.1 | 11.7% |
| | 18.2 | | |
| | 18.1 | | |

As can be seen from the results of Table 2, the substitution of glycine at the 174^{th} site of the amino acid sequence of the spoT gene with cysteine contributes to the improvement of the yield of L-threonine for the original strain producing L-threonine with either high or low yield.

### EXAMPLE 3

(1) Construction of Plasmid pKOV-yebN^{(G74A)} with yebN Gene Coding Region Having Site-Directed Mutation (G74A) (equivalent to that glycine is substituted with aspartic acid at the 25^{th} site (G25D) in a protein-coding amino acid sequence SEQ ID NO: 25, the substituted amino acid sequence being SEQ ID NO: 26)

YEBN enzyme was encoded by a yebN gene, and in an E. coli K12 strain and a derivative strain thereof (such as W3110), an ORF sequence of the wild-type yebN gene is shown in a sequence 1907402-1907968 in GenBank accession No. AP009048.1. Two pairs of primers for amplifying yebN were designed and synthesized according to the sequence, and a vector was constructed for a base G mutating to a base A at the 74^{th} site in a yebN gene coding region sequence of an original strain. The primers (synthesized by Shanghai Invitrogen Corporation) were designed as follows:

P1:
(SEQ ID NO: 27)
5′ CGGGATCCCTTCGCCAATGTCTGGATTG 3′
(the underlined part is a restriction
endonuclease cutting site BamH I);
P2:
(SEQ ID NO: 28)
5′ ATGGAGGGTGGCATCTTTAC 3′;
P3:
(SEQ ID NO: 29)
5′ TGCATCAATCGGTAAAGATG 3′;
and
P4:
(SEQ ID NO: 30)
5′ AAGGAAAAAAGCGGCCGCCAACTCCGCACTCTGCTGTA 3′
(the underlined part is a restriction endonuclease
cutting site Not I).

The construction method was as follows: using primers P1/P2 and P3/P4 for PCR amplification by taking a genome of a wild-type strain E. coli K12 as a template to obtain two DNA fragments having a length of 690 bp and 700 bp and point mutation (yebN^{G74A)}-Up and yebN^{G74A)}-Down fragments). PCR system: 10× Ex Taq buffer 5 μL, dNTP mixture (2.5 mM each) 4 μL, Mg²⁺ (25 mM) 4 μL, primers (10 pM) 2 μL each, Ex Taq (5 U/μL) 0.25 μL, total volume 50 μL, wherein the PCR was performed as follows: denaturing at 94° C. for 30 s, annealing at 52° C. for 30 s, and extending at 72° C. for 30 s (for 30 cycles). The two DNA fragments were separated and purified by agarose gel electrophoresis, and then the two purified DNA fragments were taken as templates, and P1 and P4 were taken as primers to perform overlap PCR to obtain a fragment (yebN^{(G74A)}-Up-Down) having a length of about 1340 bp.

PCR system: 10× Ex Taq buffer 5 μL, dNTP mixture (2.5 mM each) 4 μL, Mg²⁺ (25 mM) 4 μL, primers (10 pM) 2 μL each, Ex Taq (5 U/μL) 0.25 μL, total volume 50 μL, wherein the overlap PCR was performed as follows: denaturing at 94° C. for 30 s, annealing at 52° C. for 30 s, and extending at 72° C. for 60 s (for 30 cycles). The yebN^{(G74A)}-Up-Down fragment was separated and purified by agarose gel electrophoresis, then the purified fragment and a pKOV plasmid (purchased from Addgene) were digested with BamH I/Not I, and the digested yebN^{(G74A)}-Up-Down fragment and the digested pKOV plasmid were separated and purified by agarose gel electrophoresis followed by ligation to obtain a vector pKO V-yebN^{(G74A)}. The vector pKOV-yebN^{(G74A)} was sent to a sequencing company for sequencing and identification, and the vector pKOV-yebN^{(G74A)} with the correct point mutation (yebN^{(G74A)}) was stored for later use.

(2) Construction of Engineered Strain with yebN^{(G74A)} Having Point Mutation

A wild-type yebN gene was reserved on chromosomes of a wild-type Escherichia coli strain E. coli K12 (W3110) and a high-yield L-threonine production strain E. coli CGMCC 7.232 (preserved in China General Microbiological Culture Collection Center). The constructed plasmid pKOV-yebN^{(G74A)} was transferred into E. coli K12 (W3110) and E. coli CGMCC 7.232, respectively, and through allele replacement, the base G mutated to the base A at the 74^{th} site of the yebN gene sequences in the chromosomes of the two strains.

The specific process was as follows: transforming the plasmid pKO V-yebN^{(G74A)} into host bacterium competent cells through an electrotransformation process, and adding the cells into 0.5 mL of a SOC liquid culture medium; resuscitating the mixture in a shaker at 30° C. and 100 rpm for 2 h; coating an LB solid culture medium having a chloramphenicol content of 34 μg/mL with 100 μL of the culture solution, and culturing at 30° C. for 18 h; selecting grown monoclonal colonies, inoculating the colonies in 10 mL of an LB liquid culture medium, and culturing at 37° C. and at 200 rpm for 8 h; coating an LB solid culture medium having a chloramphenicol content of 34 μg/mL with 100 μL of the culture solution, and culturing at 42° C. for 12 h; selecting 1-5 single colonies, inoculating the colonies in 1 mL of an LB liquid medium, and culturing at 37° C. and 200 rpm for 4 h; coating an LB solid culture medium containing 10% of sucrose with 100 uL of the culture solution, and culturing at 30° C. for 24 h; selecting monoclonal colonies, and streaking the colonies on an LB solid culture medium having a chloramphenicol content of 34 μg/mL and an LB solid culture medium in a one-to-one correspondence manner; and selecting strains which grew on the LB solid culture medium and could not grow on the LB solid culture medium having the chloramphenicol content of 34 μg/mL for PCR amplification identification. The primers (synthesized by Shanghai Invitrogen Corporation) for use in PCR amplification were as follows:

P5:
(SEQ ID NO: 31)
5′ CCATCACGGCTTGTTGTTC 3′;
and
P6:
(SEQ ID NO: 32)
5′ ACGAAAACCCTCAATAATC 3′.

PCR system: 10× Ex Taq buffer 5 μL, dNTP mixture (2.5 mM each) 4 μL, Mg²⁺ (25 mM) 4 μL, primers (10 pM) 2 μL each, Ex Taq (5 U/μL) 0.25 μL, total volume 50 μL, wherein the PCR amplification was performed as follows: pre-denaturation at 94° C. for 5 min, (denaturing at 94° C. for 30 s, annealing at 52° C. for 30 s, and extending at 72° C. for 30 s, for 30 cycles), and over-extending at 72° C. for 10 min. SSCP (Single-Strand Conformation Polymorphism) electrophoresis was performed on the PCR-amplified product; the amplified fragment of the plasmid pKOV-yebN^{(G74A)} was taken as a positive control, the amplified fragment of the wild-type Escherichia coli was taken as a negative control, and water was taken as a blank control. In SSCP electrophoresis, single-stranded oligonucleotide chains having the same length but different sequence arrangements formed different spatial structures in an ice bath and also had different mobilities during electrophoresis. Therefore, the fragment electrophoresis position was not consistent with that of negative control, and a strain having a fragment electrophoresis position consistent with that of positive control is the successfully allele-replaced strain. PCR amplification was performed on the target fragment by taking the successfully allele-replaced strain as a template and using primers P5 and P6, and then the target fragment was ligated to a pMD19-T vector for sequencing. Through sequence comparison of a sequencing result, a recon formed by the base G mutating to the base A at the 74^{th} site in the yebN gene coding region sequence is the successfully modified strain. The recon derived from E. coli K12 (W3110) was named as YPThr05, and the recon derived from E. coli CGMCC 7.232 was named as YPThr 06.

### (3) Threonine Fermentation Experiment

The E. coli K12 (W3110) strain, the E. coli CGMCC 7.232 strain, and the mutant strains YPThr05 and YPThr06 were inoculated in 25 mL of a liquid culture medium described in Table 1, respectively, and cultured at 37° C. and 200 rpm for 12 h. Then, 1 mL of the resulting culture of each strain was inoculated in 25 mL of a liquid culture medium described in Table 1, and subjected to fermentation culture at 37° C. and 200 rpm for 36 h. The content of L-threonine was determined by HPLC, three replicates of each strain were taken, the average was calculated, and the results are shown in Table 2.

TABLE 1
Culture medium formula

| Component | Formula g/L |
|---|---|
| Glucose | 40 |
| Ammonium sulfate | 12 |
| Potassium dihydrogen phosphate | 0.8 |
| Magnesium sulfate heptahydrate | 0.8 |
| Ferrous sulfate heptahydrate | 0.01 |
| Manganese sulfate monohydrate | 0.01 |
| Yeast extract | 1.5 |
| Calcium carbonate | 0.5 |
| L-methionine | 0.5 |
| pH value adjusted with potassium | pH 7.0 |
| hydroxide | |

TABLE 2
Threonine fermentation results

| | Fermentation | Mean value | Multiple of |
|---|---|---|---|
| Strains | volume (g/L) | (g/L) | improvement |
| E. coli K12 (W3110) | 0.02 | 0.02 | — |
| | 0.03 | | |
| | 0.02 | | |
| YPThr05 | 2.2 | 2.2 | 110 |
| | 2.1 | | |
| | 2.3 | | |
| E. coli CGMCC 7.232 | 16.0 | 16.2 | — |
| | 16.3 | | |
| | 16.2 | | |
| YPThr06 | 17.6 | 17.8 | 9.9% |
| | 17.9 | | |
| | 18.0 | | |

As can be seen from the results of Table 2, the substitution of glycine at the 25^{th} site of the amino acid sequence of the yebN gene with aspartic acid contributes to the improvement of the yield of L-threonine for the original strain producing L-threonine with either high or low yield.

The examples of the present disclosure have been described above. However, the present disclosure is not limited to the above examples. Any modification, equivalent, improvement and the like made without departing from the spirit and principle of the present disclosure shall fall within the protection scope of the present disclosure.

1. A nucleotide sequence, comprising a sequence selected from the group consisting of:
i. a sequence formed by a mutation occurring at the 82^{th} base of a coding sequence of a wild-type kdtA gene shown in SEQ ID NO: 1;
ii. a nucleotide sequence formed by a mutation occurring at the 520^{th} base of a coding sequence of a spoT gene shown in SEQ ID NO: 13; and
iii. a nucleotide sequence formed by a mutation occurring at the 74^{th} base of a coding sequence of a wild-type yebN gene shown in SEQ ID NO: 23.

2. The nucleotide sequence according to claim 1, comprising a sequence selected from the group consisting of:
i. a sequence having the mutation that guanine (G) mutates to adenine (A) at the 82^{th} base in SEQ ID NO: 1;
ii. a sequence having the mutation that guanine (G) mutates to thymine (T) at the 520^{th} base in SEQ ID NO: 13; and
iii. a sequence having the mutation that guanine (G) mutates to adenine (A) at the 74^{th} base in SEQ ID NO: 23.

3. The nucleotide sequence according to claim 1, wherein the mutated nucleotide sequence is selected from the group consisting of:
i. a sequence shown in SEQ ID NO: 2;
ii. a sequence shown in SEQ ID NO: 14; and
iii. a sequence shown in SEQ ID NO: 24.

4. A recombinant protein, encoded by the nucleotide sequence according to claim 1, wherein,
preferably, an amino acid sequence of the recombinant protein is shown in SEQ ID NO: 4; or
the amino acid sequence is shown in SEQ ID NO: 16; or
the amino acid sequence is shown in SEQ ID NO: 26.

5. A recombinant vector, comprising the nucleotide sequence according to claim 1.

6. The recombinant vector according to claim 5, wherein the recombinant vector is constructed by introducing the nucleotide sequence into a plasmid.

7. A recombinant strain, comprising the nucleotide sequence according to claim 1.

8. The recombinant strain according to claim 7, wherein the recombinant strain is formed by introducing the recombinant vector comprising the nucleotide sequence into a host strain, wherein the host strain is selected from Escherichia coli; for example, the host strain is E. coli K12, a derivative strain thereof E. coli K12 (W3110), or an E. coli CGMCC 7.232 strain.

9. A construction method for the recombinant strain according to claim 7, comprising the following steps:
(1) modifying the nucleotide sequence of the wild-type gene shown in SEQ ID NO: 1 or SEQ ID NO: 13 or SEQ ID NO: 23 to obtain a mutated nucleotide sequence shown in SEQ ID NO: 2 or SEQ ID NO: 14 or SEQ ID NO: 24;
(2) ligating the mutated nucleotide sequence to a plasmid to construct a recombinant vector, preferably, the plasmid being a pKOV plasmid; and
(3) introducing the recombinant vector into a host strain to obtain the recombinant strain.

10. A method of preparing L-threonine, comprising fermenting L-threonine in presence of the nucleotide sequence according to claim 1.

11. A method of preparing L-threonine, comprising fermenting L-threonine in presence of the recombinant protein according to claim 4.

12. A method of preparing L-threonine, comprising fermenting L-threonine in presence of the recombinant vector according to claim 5.

13. A method of preparing L-threonine, comprising fermenting L-threonine in presence of the recombinant strain according to claim 7.
